(19) United States
(12) Patent Application Publication
(10) Pub. No.: US 2010/0144853 A1
(43) Pub. Date: Jun. 10, 2010

(54) NOVEL COMBINED ADMINISTRATION

(76) Inventors: Guenter Gross, Weil am Rhein (DE); Joseph Tardio, Saint Louis (FR)

Correspondence Address: HOFFMANN-LA ROCHE INC.;PATENT LAW DEPARTMENT, 340 KINGSLAND STREET, NUTLEY, NJ, 07110, US

(21) Appl. No.: 12/629,086

(22) Filed: Dec. 2, 2009

(30) Foreign Application Priority Data
Dec. 8, 2008 (EP) 08170948.7

(51) Int. Cl. A61K 31/337; A61P 9/00

(52) U.S. Cl. 514/449

(57) ABSTRACT

The present invention is directed to the combined administration of a thioester therapeutic agent (preferably of formula I) and at least one esterase inhibitor. Also provided are a pharmaceutical composition, package, and a kit comprising the aforementioned active ingredients, as well as a method for increasing the bioavailability of said thioester for the treatment and prophylaxis of a cardiovascular disorder.

### PRIORITY TO RELATED APPLICATION(S)

This application claims the benefit of European Patent Application No. 08170948.7, filed Dec. 8, 2008, which is hereby incorporated by reference in its entirety.

### BACKGROUND OF THE INVENTION

Esterases are enzymes that split esters into acids and alcohols in a chemical reaction with water. Some esterases can also split thioesters into acids and thiols, especially in the gastrointestinal tract, where degradation, formation of disulfide, or oxidation of the resulting thiol can occur before reaching the systemic circulation—thereby decreasing the bioavailability of any such thioester or thiol therapeutic agent. Esterase inhibitors inhibit the activity of esterases. Examples of esterase inhibitors include inhibitors of human carboxylesterases (Satoh et al., Chemico-Biological Interactions 162:195-211 (2006) such as Benzil derivatives, e.g., 1-{4-[oxo(phenyl)acetyl]phenyl}-2-phenylethane-1,2-dione (Wadkins et al., J. Med. Chem. 48:2906-2915 (2005), or trifluoromethyl-ketones, e.g. 3-(dodecylsulfonyl)-1,1,1-trifluoropropane-2,2-diol (Wadkins et al., Molecular Pharmacology 71:713-723 (2007), or nitrophenylester derivatives, e.g. 4-benzyl-piperidine-1-carboxylic acid 4-nitrophenyl ester, or sulfonamide derivatives, e.g. 4-chloro-N-(4-{[(4-chlorophenyl)sulfonyl]amino}phenyl)benzenesulfonamide (Wadkins et al. Molecular Pharmacology 65(6):1336-1343 (2004). Other examples of esterase inhibitors include inhibitors of lipase such as orlistat and cetilistat (Birari and Bhutani, Drug Disc. Today 12:379-389 (2007)). Orlistat (tetrahydrolipstatin, 1-(3-hexyl-4-oxo-oxetan-2-yl)tridecan-2-yl 2-formyl-amino-4-methyl-pentanoate) is commercially available in a capsule dosage form. Each capsule contains the active ingredient, orlistat, and also contains the inactive ingredients microcrystalline cellulose, sodium starch glycolate, sodium lauryl sulfate, povidone, and talc.

### SUMMARY OF THE INVENTION

The present invention is directed to the combined administration of an esterase inhibitor and a thioester. In a first aspect the present invention provides a method for increasing the bioavailability of a thioester, wherein a dosage form containing an esterase inhibitor is administered (i.e., orally) in combination with a dosage form containing the thioester.

The dosage form containing the esterase inhibitor may be administered at the same time as the dosage form containing the thioester or it may be administered in a sequence whereby the administration of the dosage form containing the esterase inhibitor is preferably prior to the administration of the dosage form containing the thioester, e.g. 1 to 60 minutes, e.g. 1 to 40 minutes, e.g. 1 to 20 minutes prior to the administration of the thioester.

The invention also provides a combination comprising: (a) a thioester or prodrug (preferably of formula I as herein described), that forms S-[2-([[1-(2-ethylbutyl)cyclohexyl]carbonyl]amino)phenyl]thiol in vivo and (2) at least one esterase inhibitor.

In another embodiment the invention provides a pharmaceutical composition comprising (a) a thioester or prodrug (preferably of formula I) that forms S-[2-([[1-(2-ethylbutyl)cyclohexyl]carbonyl]amino)phenyl]thiol in vivo, (b) at least an esterase inhibitor, and (c) one or more pharmaceutically acceptable carriers. The carriers or excipients must be acceptable in the sense of being compatible with the other ingredients and not deleterious to the recipient thereof. For example, pharmaceutically acceptable carriers or excipients are intended to include any and all material compatible with pharmaceutical administration including solvents, dispersion media, coatings, antibacterial and antifungal agents, isotonic and absorption delaying agents, and other materials and compounds compatible with pharmaceutical administration.

The invention further provides a package comprising separate dosage units, of which at least one dosage unit comprises (a) a thioester or prodrug (preferably of formula I) that forms S-[2-([[1-(2-ethylbutyl)cyclohexyl]carbonyl]amino)phenyl]thiol in vivo, and (b) at least one other dosage unit that comprises an esterase inhibitor. A “package” is understood to be any package useful for stable storage of the dosage units. The package may, for example, be a glass or plastic (e.g., a high-density polyethylene) container generally used for packaging and storage of tablets. Another form of packaging is a blister pack. Blister packs are well known in the packaging industry and are being widely used for the packaging of pharmaceutical unit dosage forms (e.g., tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via the opening.

The invention additionally provides a kit comprising (a) a first pharmaceutical composition comprising a therapeutically effective amount of (i) a thioester or prodrug (preferably of formula I) that forms S-[2-([[1-(2-ethylbutyl)cyclohexyl]carbonyl]amino)phenyl]thiol in vivo, and (ii) a pharmaceutically acceptable carrier, (b) a second pharmaceutical composition comprising (i) at least one esterase inhibitor, and (ii) a pharmaceutically acceptable carrier, (c) prescribing information, and (d) a container, wherein the first and second pharmaceutical compositions can be the same or different, and wherein the prescribing information includes advice to a patient regarding co-administration of the thioester or prodrug (preferably of formula I) that forms S-[2-([[1-(2-ethylbutyl)cyclohexyl]carbonyl]amino)phenyl]thiol in vivo, and the esterase inhibitor.

The invention additionally provides a method for the treatment or prophylaxis of a cardiovascular disorder in a patient, which comprises treating the patient with a therapeutically effective amount of a combination of (a) a thioester or prodrug (preferably of formula I) that forms S-[2-([[1-(2-ethylbutyl)cyclohexyl]carbonyl]amino)phenyl]thiol in vivo, and (b) at least one esterase inhibitor. Preferably, the esterase inhibitor is administered prior to the administration of the dosage form containing the thioester.

The cardiovascular disorders include, but are not limited to, cardiovascular disease, coronary heart disease, coronary artery disease, hypoalphalipoproteinemia (low levels of HDL cholesterol), hyperbetalipoproteinemia (high levels of LDL cholesterol), hypercholesterolemia, hyperlipidemia, and atherosclerosis. Additional cardiovascular disorders which can be treated or prevented include, but are not limited to, hypertension, hypertriglyceridemia, hyperlipoproteinemia, peripheral vascular disease, angina, ischemia, primary hypercholesterolemia (homozygous and heterozygous familial and nonfamilial), mixed dylipidemis (Frederickson Types IIa and IIb), and myocardial infarction. Following treatment with the above-described combination, the progression of atherosclerotic plaques is preferably slowed or arrested (e.g., in coronary arteries, in carotid arteries, and/or in the peripheral arterial system) in a patient. Preferably, the atherosclerotic plaques regress following treatment (e.g., in coronary arteries, in carotid arteries, and/or in the peripheral arterial system) in a patient.

Examples of thioesters include those disclosed in EP 1020439 A1, e.g. compounds of formula I:

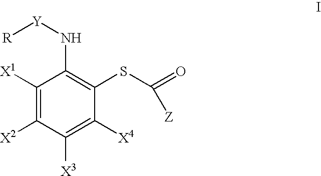

wherein:
R is selected from the group consisting of: (1) a C₁-C₁₀alkyl, (2) a C₂-C₁₀alkenyl, (3) a haloC₁-C₄alkyl, (4) a C₃-C₁₀cycloalkyl, (5) a C₅-C₈cycloalkenyl, (6) a C₃-C₁₀cycloalkylC₁-C₁₀alkyl, (7) aryl, (8) aralkyl, and (9) a 5- or 6-membered heterocyclic group having 1 to 3 nitrogen, oxygen or sulfur atoms,
X¹, X², X³ and X⁴ are independently selected from the group consisting of: (1) hydrogen, (2) halogen, (3) a C₁-C₄alkyl, (4) a haloC₁-C₄alkyl, (5) a C₁-C₄alkoxy, (6) cyano, (7) nitro, (8) acyl, and (9) aryl,

### Y is —CO— or —SO₂; and

Z is a C₁-C₁₀alkyl, a C₃-C₁₀cycloalkyl or a C₃-C₁₀cycloalkylC₁-C₁₀alkyl.

In a preferred embodiment of the present invention the thioester is thioisobutyric acid S-(2-{[1-(2-ethyl-butyl)-cyclohexanecarbonyl]-amino}-phenyl)ester.

The thioester may be formulated in any conventional formulation including capsule formulations or tablet formulations. An example of a thioester tablet formulation is disclosed in WO2004/082593 and given in Table 1.

TABLE 1
Example of a tablet formulation of COMPOUND A

| Ingredient | amount (mg) | content (%) |
|---|---|---|
| Thioester (COMPOUND A) | 300 | 54.8 |
| Hydroxypropyl methylcellulose 2910 | 18 | 3.3 |
| Crospovidone | 119.8 | 21.9 |
| Talc | 18 | 3.3 |
| Low-substituted Hydroxypropyl cellulose | 90 | 16.5 |
| Magnesium stearate | 1.2 | 0.2 |
| Total | 547 | 100.0 |

The combined oral administration of an esterase inhibitor and a thioester provides for an increase in stability and the bioavailability of the thioester in the gastrointestinal tract of patients which is shown by the following test results:

### EXAMPLE 1

### Stability of thioisobutyric acid S-(2-{[1-(2-ethyl-butyl)-cyclohexanecarbonyl]-amino}-phenyl)ester

### Stock Solutions:

(a) FeSSIF (Fed State Simulated Intestinal Fluid) and phosphate buffered saline solution:

| | phosphate buffered | | Pro-FeSSIF | | Concentrate | | |
|---|---|---|---|---|---|---|---|
| Ingredient | saline solution | | 1.11x | | (FeSSIF 10x) | | FeSSIF |
| Sodium chloride | 3.2 | g | 6.6 | g | | | 202 mM |
| Sodium dihydrogen- | 2.0 | g | | | | | — |
| phosphate dihydrate | | | | | | | |
| Acetic acid | | | 4.8 | g | | | 144 mM |
| Sodium taurocholate | | | | | 806 | mg | 15 mM |
| Lecithin | | | | | 300 | mg | 3.75 mM |
| Sodium hydroxide 1N | ad pH 6.5 | | ad pH 6.5 | | | | pH 6.5 |
| Water to total volume of | 500 | mL | 500 | mL | 10 | mL | |

FeSSEF = 1 part concentrate + 9 parts Pro-FeSSIF

(b) Stock A=drug solution (0.5 mg/mL):

5 mg of thioisobutyric acid S-(2-{[1-(2-ethyl-butyl)-cyclohexanecarbonyl]-amino}-phenyl)ester (COMPOUND A) were dissolved in 50 μL ethanol and 950 μL mixed micelles Soybean lecithin/Sodium glycocholate (158/97 mg/mL) in water were added. One part of the mixture was diluted with 9 parts of phosphate buffered saline solution at pH 6.5.

(c) Stock B=esterase inhibitor solution (1.0 mg/mL)

5 mg 1-(3-hexyl-4-oxo-oxetan-2-yl)tridecan-2-yl 2-formyl-amino-4-methyl-pentanoate (COMPOUND B) were dissolved in 250 μL ethanol. 4.75 mL mixed micelles soybean lecithin/sodium glycocholate (30/80.6 mg/mL) were added to give a solution of 1 mg/mL COMPOUND B.

(d) Stock C=Lipase in phosphate buffered saline solution (PBS) pH 6.5 (example given at 24 mg/mL, with a final enzyme activity of about 450 U/mL)

240 mg Lipase (Lipase from Hog Pancreas, 20.6 U/mg Lipase, CAS No. 9001-62-1, Fluka, Art. No. 62300) were weighed into a vial and dissolved by stirring in 10 mL phosphate buffered saline solution pH 6.5. Any undissolved material was removed by centrifugation during 5 min at 5000 rpm. The supernatant was used for the experiments and diluted with PBS to obtain the Lipase at the final concentration of 0.55 mg/mL.

200 μL of 1-(3-hexyl-4-oxo-oxetan-2-yl)tridecan-2-yl 2-formyl-amino-4-methyl-pentanoate solution (stock B) were injected in 1.6 mL Lipase solution (stock C) (from stock C to obtain a final enzyme concentration of 0.55 mg/mL). To this, 200 μL of drug solution (stock A) were injected at time zero. The mixture was incubated at 37° C., while mixing gently with a rotating shaker at about 2 rpm. At regular intervals 200 μL samples were taken and diluted immediately with 600 μL of isopropanol. The combined solution/suspension was centrifuged during 5 min at 3000 rpm and the concentration of COMPOUND A as well as its potential degradation products were determined in the supernatant by HPLC analysis.

To obtain the value at time zero, 600 μL of isopropanol were added to 180 μL of a 0.55 mg/mL Lipase in a phosphate buffered saline solution to inhibit the enzymes followed by the addition of 20 μL of drug solution (stock A).

| Time | +100 μg/mL COMPOUND B | | w/o COMPOUND B | |
|---|---|---|---|---|
| (min) | COMPOUND A | Disulfide | COMPOUND A | Disuffide |
| 0 | 46.9 | | | |
| 5 | 48.2 | <0.2 | 27.2 | 16.4 |
| 10 | 48.2 | <0.2 | 14.7 | 26.8 |
| 15 | 48.2 | <0.2 | 7.3 | 33.1 |
| 20 | 48.0 | <0.2 | 4.1 | 36.3 |
| 30 | 48.3 | <0.2 | <0.1 | 38.0 |
| 60 | 40.1 | <0.2 | <0.1 | 32.3 |

Concentration in μg/mL of COMPOUND A (theoretical concentration is 50 μg/mL) and Disulfide with 0.55 mg/mL (11 U/mL) Lipase

### EXAMPLE 2

### Comparison of different experimental procedures and influence of the lipase inhibitor concentration on stability of thioisobutyric acid S-(2-{[1-(2-ethyl-butyl)-cyclohexanecarbonyl]-amino}-phenyl)ester

### Procedure A (Premix of Lipase and COMPOUND B, Followed by Addition of Compound A):

1600 μL of Lipase in phosphate buffered saline solution (from stock C) to obtain a final enzyme concentration of 0.55 mg/mL) were mixed with 200 μL of COMPOUND B (stock B) and pre-incubated during 5 min at 37° C. At time zero 200 μL of drug solution (stock A) were added. The mixture was incubated at 37° C. After 10 min, the reaction was stopped by dilution of one part of the mixture with 3 parts of isopropanol.

### Procedure B (Premix of COMPOUND B and COMPOUND A, Followed by Addition of Lipase):

100 μL of drug solution (stock A) were mixed with 0, 12, 25 or 50 μL esterase inhibitor solution (stock B) and 100, 88, 75 or 50 μL of FeSSIF concentrate (Lecithin37.5 mM and Sodium taurocholate 150 mM) were added to make up 200 μL of a solution with COMPOUND B concentrations of 0, 12, 25 or 50 μg/mL. At time zero, 800 μL of Lipase in phosphate buffered saline solution (from stock C to obtain final enzyme concentrations of 0.55 mg/mL) were added. The reaction mixture was incubated at 37° C. and stopped after 10 minutes by diluting one part of the reaction mixture with 3 parts Isopropanol.

| | Procedure A | | Procedure B | |
|---|---|---|---|---|
| | (Lipase + COMPOUND B, | | (COMPOUND B + COMPOUND A, | |
| | then COMPOUND A) | | then Lipase) | |
| COMPOUND B | COMPOUND A | SD | COMPOUND A | SD |
| (μg/mL) | (μg/mL) | (n = 3) | (μg/mL) | (n = 3) |
| 0 | 14.7 | n.d. | 23.0 | 1.0 |
| 12 | n.d. | n.d. | 46.4 | 0.6 |
| 25 | n.d. | n.d. | 46.5 | 1.0 |
| 50 | 46.4 | 0.7 | 46.1 | 1.1 |
| 100 | 48.2 | 0.8 | n.d. | n.d. |

Concentration in μg/mL of COMPOUND A (theoretical concentration of 50 μg/ml) with 0.55 mg/mL (11 U/mL) of Lipase

### Procedure B′ (Premix of COMPOUND B and COMPOUND A, Followed by Addition of Lipase):

The tests were repeated as described under procedure B but with final COMPOUND B concentrations of 0, 3, 6, 12 μg/ml.

| COMPOUND B (μg/ml) | COMPOUND A (μg/ml) |
|---|---|
| 0 | 14.2 |
| 3 | 45.9 |
| 6 | 46.5 |
| 12 | 45.7 |

Concentration in μg/mL of COMPOUND A (theoretical concentration is 50 μg/mL) after 10 min incubation with 0.55 mg/mL (11 U/mL) of Lipase

### Procedure C (Premix of COMPOUND A and Lipase, Followed by Addition of COMPOUND B):

Remark: Final COMPOUND B concentration: 3 μg/ml

### 1^{st} Experiment:

220 μL of drug solution (stock A) and 220 μL of FeSSIF concentrate (Lecithin 37.5 mM and Sodium taurocholate 150 mM) were mixed. At time zero the reaction was started by addition of 1700 μl of Lipase in phosphate buffered saline solution (from stock C to obtain a final enzyme concentration of 0.55 mg/mL). The mixture was incubated at 37° C. and after 5 min, a 200 μL sample was taken for HPLC analysis. Then 60 μL of COMPOUND B solution (stock B) previously diluted 1:10 in phosphate buffered saline solution (corresponds to 100 μg/mL COMPOUND B) was added and as a function of time, samples of 200 μL were taken, diluted immediately with 600 μL of isopropanol and centrifuged for 5 min at 3000 rpm. The supernatant was analyzed by HPLC.

### 2^{nd} Experiment (Identical to Experiment 1 but Different Order of Reagent Addition):

1700 μl of Lipase in phosphate buffered saline solution (from stock C to obtain a final enzyme concentration of 0.55 mg/mL) were mixed with 220 μL of FeSSIF concentrate and at time zero 220 μL of drug solution (stock A) was added. After 7 min of incubation, a 200 μL sample was taken, 60 μL of diluted COMPOUND B solution (stock B) was added and continued as described for the 1^{st} experiment.

| Time | +3 μg/mL COMPOUND B at T = 5 min | | +3 μg/mL COMPOUND B at T = 7 min | |
|---|---|---|---|---|
| (min) | COMPOUND A | Disulfide | COMPOUND A | Disulfide |
| 0 | 45.3 | | 45.3 | |
| 5 | 41.5 | 5.3 | 40.4 | 6.6 |
| 10 | 39.1 | 5.8 | 38.4 | 7.6 |
| 15 | 39.3 | 5.6 | 38.2 | 7.2 |
| 20 | 39.1 | 5.6 | 37.9 | 7.6 |
| 25 | 39.3 | 5.8 | 37.8 | 7.6 |
| 30 | 39.3 | 5.8 | 37.9 | 7.2 |

Concentration in μg/mL of COMPOUND A (theoretical concentration is 50 μg/mL) and Disulfide after incubation with 0.55 mg/mL (11 U/mL) of Lipase

By these experiments it could be demonstrated that the lipase inhibitor COMPOUND B efficiently prevents lipase induced hydrolytic degradation of COMPOUND A. The presence of the esterase inhibitor effectively maintained the stability of COMPOUND A and inhibited formation of disulfide.

### EXAMPLE 3

### Influence of the lipase inhibitor concentration on stability of thioisobutyric acid S-(2-{[1-(2-ethyl-butyl)-cyclohexanecarbonyl]-amino}-phenyl)ester in human intestinal fluids

### Procedure D (COMPOUND A and COMPOUND B in Solution):

Fasted Intestinal Fluids: the fluids from 4 different subjects were collected with a naso-jejunal tube after fasting overnight and pooled.

Stimulated Intestinal Fluids: the fluids from 3 different subjects were collected after fasting overnight with a nasojejunal tube and pooled. For the stimulation, twenty minutes before the beginning of the collection, volunteers were provided with chewing-gums and were asked to chew over a 15 minutes period, maintaining the taste by replacing the sticks of gum approximately every 2 minutes.

### Stock Solutions

(a) Stock D=drug solution (5 mg/mL):

5 mg of thioisobutyric acid S-(2-{[1-(2-ethyl-butyl)-cyclohexanecarbonyl]-amino}-phenyl)ester (COMPOUND A) were dissolved in 50 μL ethanol and 950 μL mixed micelles solution containing Soybean lecithin/Sodium glycocholate (158/97 mg/mL) in water were added.

(b) Stock E=esterase inhibitor solution (0.3 mg/mL)

3 mg of 1-(3-hexyl-4-oxo-oxetan-2-yl)tridecan-2-yl 2-formyl-amino-4-methyl-pentanoate (COMPOUND B) were dissolved in 1 mL of ethanol. 100 μL of this solution was diluted with 900 μL of FeSSIF concentrate (10×).

(c) Stock F=premix of COMPOUND A and COMPOUND B (respectively 500 and 30 μg/mL)

100 μL of drug solution (stock D) were mixed with 100 μL of esterase inhibitor solution (stock E) and 800 μL of pro-FaSSIF were added.

(d) Stock G=premix of COMPOUND A and COMPOUND B (respectively 500 and 10 μg/mL)

100 μL of premix (stock F) were mixed with 200 μL of drug solution (stock A).

(e) Stock H=premix of COMPOUND A and COMPOUND B (respectively 500 and 20 μg/mL)

200 μL of premix (stock F) were mixed with 100 μL of drug solution (stock A). 315 μL of intestinal fluids (fasted and stimulated) were pre-heated at 37° C. for about 5 min. At time zero, 35 μL of premix solutions were added, i.e. either stock A, stock G, stock H or stock F, to obtain 50 μg/mL of COMPOUND A and final concentrations of COMPOUND B of 0, 1, 2 or 3 μg/mL. The samples were incubated at 37° C. while gently stirring. The reaction was stopped after 2 min or after 10 min by dilution of 1 part of the reaction mixture with 3 parts of isopropanol and the resulting mixture was centrifuged for 5 min at 5′000 rpm. The supernatant was analyzed by HPLC.

| COMPOUND B | COMPOUND A (μg/mL) | | Disulfide (μg/mL) | |
|---|---|---|---|---|
| conc. (μg/mL) | 2 min | 10 min | 2 min | 10 min |
| 0 | 0.48 | 0.48 | 31.56 | 32.68 |
| 1 | 18.00 | 1.58 | 15.43 | 21.53 |
| 2 | 36.91 | 28.59 | 7.75 | 10.66 |
| 3 | 43.72 | 37.24 | 5.16 | 6.81 |

Concentration in (μg/mL of COMPOUND A (theoretical concentration is 50 μg/mL) and Disulfide in intestinal fluid obtained from fasted subjects

| COMPOUND B | COMPOUND A (μg/mL) | | Disulfide (μg/mL) | |
|---|---|---|---|---|
| conc. (μg/mL) | 2 min | 10 min | 2 min | 10 min |
| 0 | 0.48 | 0.48 | 39.95 | 40.16 |
| 1 | 26.06 | 2.76 | 18.82 | 28.59 |
| 2 | 38.92 | 24.93 | 10.79 | 14.95 |
| 3 | 46.91 | 40.48 | 8.33 | 10.36 |

Concentration in μg/mL of COMPOUND A (theoretical concentration is 50 μg/mL) and Disulfide in intestinal fluid obtained from subjects after stimulation

By the above experiments it is evident that the presence of COMPOUND B stabilizes dissolved COMPOUND A towards hydrolysis in intestinal fluids and reduces the formation of the disulfide.

### EXAMPLE 4

### Influence of the lipase inhibitor concentration on stability of thioisobutyric acid S-(2-{[1-(2-ethyl-butyl)-cyclohexanecarbonyl]-amino}-phenyl)ester in human intestinal fluids

### Procedure E (COMPOUND A and COMPOUND B in Suspension, Excess Compound Separated by Centrifugation):

Stock J=COMPOUND B suspension (2 mg/mL)

100 μL of FeSSIF concentrate and 900 μL of pro-FeSSIF pH6.5 were added to 4 mg commercially available Orlistat formulation (Xenical®) containing 2 mg of lipase inhibitor 1-(3-hexyl-4-oxo-oxetan-2-yl)tridecan-2-yl 2-formyl-amino-4-methyl-pentanoate (COMPOUND B) in a vial and the formulation was dispersed by gently stirring during 15 min to obtain a fine homogenous suspension.

500 μL of freshly thawed intestinal fluids (fasted and stimulated state) were added to 1 mg of COMPOUND A in a 1 mL glass vial immediately followed by the addition of either 0, 10 or 20 μL of COMPOUND B suspension (stock J) corresponding to 0, 40 or 80 μg/mL of COMPOUND B. The amount of COMPOUND B was 0, 2% or 4% COMPOUND B relative to COMPOUND A. The mixture was incubated at 37° C. with gentle stirring. Samples of 90 μL were removed as function of time and centrifuged at 13′000 rpm (about 12′000×g) for 5 min. 50 μL of the supernatant was diluted with 150 μL isopropanol, centrifuged at 5′000 rpm for 5 min and the clear supernatant was analyzed by HPLC.

| | | HIF-fasted (μg/mL) | | | HIF-stimulated (μg/mL) | | |
|---|---|---|---|---|---|---|---|
| Time | | COMPOUND A | Thiophenol | Disulfide | COMPOUND A | Thiophenol | Disulfide |
| 15 | min | 1.62 | 0.00 | 218.12 | 3.09 | 2.55 | 49.80 |
| 30 | min | 1.86 | 3.41 | 514.94 | 0.52 | 24.33 | 158.53 |
| 1 | h | 0.49 | 29.55 | 994.29 | 0.62 | 89.14 | 348.73 |
| 2 | h | 0.49 | 125.39 | 1260.90 | 0.82 | 149.88 | 500.57 |
| 4 | h | 0.49 | 151.84 | 1313.31 | 1.31 | 208.98 | 619.27 |

Concentration in μg/mL of COMPOUND A (Thioester), Thiophenol and Disulfide in fasted and stimulated human intestinal fluids (HIF) without COMPOUND B.

| | | HIF-fasted (μg/mL) | | | HIF-stimulated (μg/mL) | | |
|---|---|---|---|---|---|---|---|
| Time | | COMPOUND A | Thiophenol | Disulfide | COMPOUND A | Thiophenol | Disulfide |
| 15 | min | 1.12 | 0.16 | 110.57 | 0.74 | 0.16 | 40.71 |
| 30 | min | 11.75 | 3.85 | 363.56 | 0.55 | 0.89 | 102.65 |
| 1 | h | 111.05 | 13.54 | 646.50 | 10.53 | 75.39 | 343.60 |
| 2 | h | 217.35 | 25.60 | 902.02 | 32.71 | 149.23 | 565.70 |
| 4 | h | 281.66 | 64.21 | 1075.49 | 221.31 | 349.31 | 764.51 |

Concentration in μg/mL of COMPOUND A (Thioester), Thiophenol and Disulfide in fasted and stimulated human intestinal fluids (HIF) with 40 μg/mL of COMPOUND B.

| | | HIF-fasted (μg/mL) | | | HIF-stimulated (μg/mL) | | |
|---|---|---|---|---|---|---|---|
| Time | | COMPOUND A | Thiophenol | Disulfide | COMPOUND A | Thiophenol | Disulfide |
| 15 | min | 5.62 | 0.16 | 31.75 | 1.00 | 0.48 | 54.34 |
| 30 | min | 16.36 | 0.16 | 95.68 | 11.80 | 4.51 | 115.01 |
| 1 | h | 89.60 | 2.11 | 281.19 | 64.63 | 6.38 | 211.64 |
| 2 | h | 230.34 | 8.13 | 458.77 | 131.64 | 7.64 | 291.49 |
| 4 | h | 336.00 | 12.61 | 578.53 | 171.25 | 7.11 | 345.98 |

Concentration in μg/mL of COMPOUND A (Tnioester), Thiophenol and Disulfide in fasted and stimulated human intestinal fluids (HIF) with 80 μg/mL of COMPOUND B.

The above experiments show that the addition of COMPOUND B in the form of a suspension stabilizes the thioester which is present in excess in intestinal fluids against hydrolysis and subsequent oxidation of the thiol formed.

### EXAMPLE 5

### Influence of the lipase inhibitor concentration on stability of thioisobutyric acid S-(2-{[1-(2-ethyl-butyl)-cyclohexanecarbonyl]-amino}-phenyl)ester in human intestinal fluids

Procedure F (COMPOUND A and COMPOUND B in suspension, the degradation immediately stopped without separation of excess compound):
Stock K=COMPOUND B suspension (0.5 mg/mL)

Dilute 1 part of COMPOUND B suspension (stock J) with 3 parts of FeSSIF pH6.5 solution.

500 μL of freshly thawed intestinal fluids (fasted and stimulated state) were added to 1.2 mg of COMPOUND A in a 1 mL glass vial. The suspended compound was stirred for 15 min at 37° C. to obtain wetting and a homogenous suspension. In parallel, 4 glass vials were prepared with 20 μL of a COMPOUND B suspension at different concentrations. These concentrations were obtained by mixing FeSSIF pH6.5 and COMPOUND B suspension (stock K) in different ratios, respectively 1:0, 1:3, 1:1, 0:1 to obtain 0, 1, 2 and 4% related to COMPOUND A. At time zero, 100 μL of COMPOUND A suspension preincubated at 37° C. were added to the 20 μL COMPOUND B suspension. The mixture was incubated at 37° C. with gentle stirring. After 15 min and 60 min, the reaction was stopped by mixing a 50 μL sample with 150 μL isopropanol and centrifuged at 5′000 rpm during 5 min. The clear supernatant was analyzed for the hydrolysis product and its oxidized form by HPLC.

| COMPOUND | T = 15 min | | | T = 60 min | | |
|---|---|---|---|---|---|---|
| B conc. | COMPOUND | Thiophenol | Disulfide | COMPOUND | Thiophenol | Disulfide |
| (μg/mL) | A (μg/mL) | (μg/mL) | (μg/mL) | A (μg/mL) | (μg/mL) | (μg/mL) |
| 0 | 1111.92 | 12.07 | 512.32 | 182.50 | 22.18 | 1004.67 |
| 20 | 1299.01 | 9.58 | 367.37 | 718.26 | 15.27 | 569.98 |
| 40 | 1381.23 | 5.42 | 290.53 | 598.81 | 8.36 | 331.72 |
| 80 | 1728.95 | 6.34 | 290.06 | 826.93 | 10.49 | 299.09 |

Concentration in μg/mL of Compound A, Thiophenol and Disulfide in fasted human intestinal fluids (HIF) with 0, 20, 40 and 80 μg/mL of COMPOUND B corresponding to 0, 1, 2 and 4% with regard to the Thioester concentration.

| COMPOUND | T = 15 min | | | T = 60 min | | |
|---|---|---|---|---|---|---|
| B conc. | COMPOUND | Thiophenol | Disulfide | COMPOUND | Thiophenol | Disulfide |
| (μg/mL) | A (μg/mL) | (μg/mL) | (μg/mL) | A (μg/mL) | (μg/mL) | (μg/mL) |
| 0 | 1569.58 | 68.88 | 392.71 | 400.63 | 412.83 | 903.76 |
| 20 | 2034.38 | 13.26 | 252.67 | 1503.21 | 29.88 | 459.09 |
| 40 | 2163.01 | 8.30 | 215.76 | 1515.41 | 20.48 | 269.78 |
| 80 | 1945.50 | 5.58 | 186.77 | 1449.03 | 8.51 | 212.28 |

Concentration in μg/mL of Compound A, Thiophenol and Disulfide in fasted human intestinal fluids (HIF) with 0, 20, 40 and 80 μg/mL of COMPOUND B corresponding to 0, 1, 2 and 4% with regard to the Thioester concentration.

By these experiments it could be demonstrated that the esterase inhibitor COMPOUND B efficiently prevents gastrointestinal enzyme induced hydrolytic degradation of COMPOUND A.

1. A method for increasing the bioavailability of a thioester, wherein a dosage form containing an esterase inhibitor is administered in combination with a dosage form containing the thioester.

2. The method according to claim 1 wherein the dosage form containing the esterase inhibitor is administered at the same time as the dosage form containing the thioester.

3. The method according to claim 1 wherein the dosage form containing the esterase inhibitor is administered prior to the administration of the dosage form containing the thioester.

4. The method according to claim 3, wherein the dosage form containing the esterase inhibitor is administered 1 to 60 minutes prior to the administration of the dosage form containing the thioester.

5. The method according to claim 1 wherein the thioester is thioisobutyric acid S-(2-{[1-(2-ethyl-butyl)-cyclohexanecarbonyl]-amino}-phenyl)ester.

6. The method according to claim 5 wherein the esterase inhibitor is 1-(3-hexyl-4-oxo-oxetan-2-yl)tridecan-2-yl 2-formyl-amino-4-methyl-pentanoate.

7. A method according to claim 1 wherein the ratio of an esterase inhibitor to thioester is between 1:100 and 1:5.

8. A composition, package, or kit comprising (a) a thioester that forms S-[2-([[1-(2-ethylbutyl)cyclohexyl]carbonyl]amino)phenyl]thiol in vivo and (b) at least an esterase inhibitor.

9. A pharmaceutical composition, package, or kit comprising:
(a) a thioester of formula I: 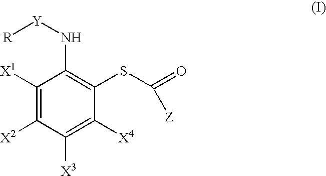
wherein:
R is selected from the group consisting of: (1) a C₁-C₁₀alkyl, (2) a C₂-C₁₀alkenyl, (3) a haloC₁-C₄alkyl, (4) a C₃-C₁₀cycloalkyl, (5) a C₅-C₈cycloalkenyl, (6) a C₃-C₁₀cycloalkylC₁-C₁₀alkyl, (7) aryl, (8) aralkyl, and (9) a 5- or 6-membered heterocyclic group having 1 to 3 nitrogen, oxygen or sulfur atoms,
X¹, X², X³ and X⁴ are independently selected from the group consisting of: (1) hydrogen, (2) halogen, (3) a C₁-C₄alkyl, (4) a haloC₁-C₄alkyl, (5) a C₁-C₄alkoxy, (6) cyano, (7) nitro, (8) acyl, and (9) aryl,
Y is —CO— or —SO₂;
Z is a C₁-C₁₀alkyl, a C₃-C₁₀cycloalkyl or a C₃-C₁₀cycloalkylC₁-C₁₀alkyl; and
(b) an esterase inhibitor which is 1-(3-hexyl-4-oxo-oxetan-2-yl)tridecan-2-yl 2-formyl-amino-4-methyl-pentanoate.

10. The pharmaceutical composition, package, or kit of claim 9, comprising: (a) thioisobutyric acid S-(2-{[1-(2-ethyl-butyl)-cyclohexanecarbonyl]-amino}-phenyl)ester; and (b) 1-(3-hexyl-4-oxo-oxetan-2-yl)tridecan-2-yl 2-formyl-amino-4-methyl-pentanoate.

11. The pharmaceutical composition, package, or kit of claim 10 further comprising one or more pharmaceutically acceptable carriers.

12. A method for the treatment or prophylaxis of a cardiovascular disorder in a patient, which comprises treating the patient with a therapeutically effective amount of a combination of: (a) a thioester that forms S-[2-([[1-(2-ethylbutyl)cyclohexyl]carbonyl]amino)phenyl]thiol in vivo, and (b) at least one esterase inhibitor.

13. The method of claim 12, which comprises treating the patient with a therapeutically effective amount of a combination of: (a) thioisobutyric acid S-(2-{[1-(2-ethyl-butyl)-cyclohexanecarbonyl]-amino}-phenyl)ester; and (b) 1-(3-hexyl-4-oxo-oxetan-2-yl)tridecan-2-yl 2-formyl-amino-4-methyl-pentanoate.
